(19) United States
(12) Patent Application Publication
(10) Pub. No.: US 2006/0198886 A1
(43) Pub. Date: Sep. 7, 2006

(54) Medicament having coated methenamine combined with acidifier

(76) Inventor: Richard B. Jenkins, Tampa, FL (US)

Correspondence Address: WYATT BARTON PRATT, 15 COLUMBIA AVENUE, HOPEWELL, NJ, 08525, US

(21) Appl. No.: 11/068,712

(22) Filed: Mar. 1, 2005

(51) Int. Cl. A61K 9/22; A61K 31/205; A61K 38/04

(52) U.S. Cl. 424/468; 514/19; 514/554

(57) ABSTRACT

A solid pharmaceutical medicament has a coated methenamine compound in combination with an urinary acidifier.

### BACKGROUND OF THE INVENTION

1. Field of the Invention

The present invention pertains to solid pharmaceutical combinations of a coated methenamine compound combined with an acidifier.

2. Brief Description of the Related Art

Methenamine, also known as hexamethylenetetramine or HMTA, is a compound useful in the treatment of urinary infections. Typically, the methenamine mandelate is administered with sodium acid phosphate monohydrate to increase the formation of formaldehyde from hydrolysis of the methenamine mandelate in the urine to impart an antiseptic effect. At a pH of 6.0 or above, and particularly in an alkaline medium, methenamine is devoid of antiseptic action.

Methenamine has a wide spectrum bacteriacidal and bacteriostatic activity and low toxicity, however, it requires a constant acidic urine to induce the continuous yield of formaldehyde. The separate concomitant administration of an acidifying agent for urine necessitates frequent pH assays of the urine and a constant reminder to the patient to take the second, acidifying agent. This results in a haphazard coadministration of the acidifying agent along with the methenamine.

There is a need in the art to provide improved pharmaceutical formulations of methenamine compounds as an antiseptic agent. The present invention addresses this and other needs.

### SUMMARY OF THE INVENTION

The present invention includes a solid pharmaceutical medicament comprising a coated methenamine compound effective to delay release of the methenamine within the gastrointestinal tract, particularly after passage through the stomach, in combination with an acidifier effective for acidifying urine to a pH of from about 6 or less. The medicament may be administered in unit dosage form. Additionally, the medicament may include a second active pharmaceutical ingredient such as an antiseptic agent.

The present invention also includes a method for urinary antiseptic treatment comprising the steps of providing the previously described medicament and dosing a patient with the medicament.

### DETAILED DESCRIPTION OF THE INVENTION

The present invention includes a solid pharmaceutical medicament having a coated active pharmaceutical ingredient of a methenamine compound in combination with an acidifier that is effective to acidify the urine of a patient to a pH of from about 6 or less. By separately coating the methenamine, the present invention provides a vehicle to protect the methenamine at different stages in the digestive system from the acidifier. It also reduces gastic pain resulting from the formation of formaldehyde with the conversion of the methenamine within the stomach. This permits greater utility of the medicament for patient usage.

When used herein, the terms active agent, active pharmaceutical ingredient, pharmaceutical actives, API, active compound, therapeutic agent, therapeutic ingredient, therapeutic compound and other like terms are used interchangeably and include salts and other pharmaceutical forms of the detailed compounds.

The methenamine compounds of the present invention include methenamine, pharmaceutically acceptable salts of methenamine and combinations thereof. Salts of the present invention include those refers to derivatives of the methenamine having the therapeutic compound modified by reacting it with an acid or base as needed to form an ionically bound pair. Examples of pharmaceutically acceptable salts include conventional non-toxic salts or the quaternary ammonium salts of the parent compound formed, for example, from non-toxic inorganic or organic acids. Suitable non-toxic salts include those derived from inorganic acids such as hydrochloric, hydrobromic, sulfuric, sulfonic, sulfamic, phosphoric, nitric and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and others known to those of ordinary skill in the art. The pharmaceutically acceptable salts of the present invention can be synthesized from the parent therapeutic compound which contains a basic or acidic moiety by conventional chemical methods. Salts of methenamine include combinations, condensation products or other salt formations of methenamine, as known in the art, such as tannin (as disclosed in U.S. Pat. No. 607,172 to Hock), quinic acid (as disclosed in U.S. Pat. No. 690,804 to Wichmann et al.), sodium acetate double salt (as disclosed in U.S. Pat. No. 852,993 to Bergell), mandelic acid (as disclosed in U.S. Pat. No. 2,124,321 to Tisza), aromatic acids (as disclosed in U.S. Pat. No. 2,179,618 to Edelman et al.), benzene monocarboxylic acids (as disclosed U.S. Pat. No. 2,912,435 to Scholz), hydroxamate compounds such as acetohydroxamic acid (see e.g., U.S. Pat. No. 4,146,644 to Griffith et al.), methenamine mandelate, hexamethylenetetramine hippurate (see e.g., U.S. Pat. No. 3,004,026 to Galat), and/or specialized enantiomorph forms (see e.g., U.S. Pat. No. 4,001,231 to Diamond) and the like. Preferred methenamine compounds include methenamine, methenamine mandelate, methenamine hippurate and combinations thereof, with methenamine mandelate most preferred.

The medicament of the present invention includes an appropriate amount of methenamine compound, generally having an amount that is therapeutically effective in a convenient dosage unit for a given patient. Preferred amounts of methenamine compound contained within the pharmaceutical formulation of the present invention may be administered, for example without limitation, in unit dosages with typical dosing amounts of the methenamine compound ranging from about 2 grams or less, such as about 1 gram to 500 milligrams (mg) or less including preferred dosages of about 200 mg to about 500 mg, more preferably from about 300 mg to about 500 mg, still more preferably from about 400 mg to about 500 mg and most preferably from about 500 mg.

The methenamine compound of the present invention is coated with a pharmaceutical glaze to delay the breakdown of the methenamine in the stomach, and preferably after leaving the stomach. The enteric coating is resistant to disintegration at low pH levels and provides drug release properties at higher pHs, such as for example becoming soluble at pH 5.5 and above, with maximum solubility rates at pHs greater than 6.5. Numerous enteric coated and/or extended release pharmaceutical compositions and the methods of making these compositions are known in the art. By using the enteric coating, delivery of the methenamine compound active pharmaceutical ingredient is delivered within the body substantially beyond the stomach in the digestive tract, e.g., delivery in the lower GI tract, i.e., in the colon, or the upper intestines, i.e., the duodenum of the small intestine. The medicament includes a coating on the methenamine compound to protect the methenamine compound and release the methenamine compound at specific locations within the digestive tract of the patient. Preferably this includes an enteric coating for release of the methenamine compound past the stomach area. The enteric coat may include one or more materials that remain intact during the period of time that the tablet resides in the stomach and do not dissolve, disintegrate, or change structural integrity in the stomach. Preferably, the methenamine compound of the present invention includes a delayed-release methodology such as that described in Pharmaceutical Dosage Forms and Drug Delivery Systems. “Modified-Release Dosage Forms and Drug Delivery Systems”, Lippincott Williams & Wilkins, 1999, Chapter 8, pp. 229-244, the disclosure of which is herein incorporated by reference in its entirety. As described therein, a delayed-release form provides is designed to release the drug from the dosage from at a time other than promptly after administration. Representative materials include keratin, keratin sandarac-tolu, salol (phenyl salicylate), salol beta-naphthylbenzoate and acetotannin, salol with balsam of Peru, salol with tolu, salol with gum mastic, salol and stearic acid, and salol and shellac, formalized protein, formalized gelatin, and formalized cross-linked gelatin and exchange resins, myristic acid-hydrogenated castor oil-cholesterol, stearic acid-mutton tallow, stearic acid-balsa mn of tolu, and stearic acid-castor oil, shellac, ammoniated shellac, ammoniated shellac-salol, shellac-wool fat, shellac-acetyl alcohol, shellac-stearic acid-balsam of tolu, and shellac n-butyl stearate, abietic acid, methyl abictate, benzoin, balsam of tolu, sandarac, mastic with tolu, and mastic with tolu, mastic with acetyl alcohol, acrylic resins such as anionic polymers synthesized from methacrylate acid and methacrylic acid methyl ester, copolymeric acrylic resins of methacrylic and methacrylic acid and methacrylic acid alkyl esters, copolymers of alkacrylic acid and alkacrylic acid alkyl esters, acrylic resins such as dimethylaminoethylmethacrylate-butylmethacrylate-methyhnethacrylate copolymer of 150,000 molecular weight, methacrylic acid-methylmethacrylate 50:50 coploymer of 135,000 molecular weight, methacrylic acid-methylmethacrylate-30:70-copolymer of 135,000 mol. wt., methacrylic acid-dimethylaminoethyl-methacrylate-ethylacrylate of 750,000 mol. wt., methacrylic acid-methylmethacrylate-ethylacrylate of 1,000,000 mol. wt., and ethylacrylate-methylmethacrylate-ethylacrylate of 550,000 mol. wt. The coating is non-toxic and preferably includes any pharmaceutically acceptable enteric polymer that is predominantly soluble in the intestinal fluid, but substantially insoluble in the gastric juices. A wide variety of other polymeric materials are known to possess various solubility properties. Representative examples of enteric polymers include, without limitation, polyvinyl acetate phthalate (PVAP), hydroxypropylmethyl-cellulose acetate succinate (HPMCAS), cellulose acetate phthalate (CAP) commercially available under the tradename Aquaten™, methacrylic acid copolymer, hydroxy propyl methylcellulose succinate, cellulose acetate succinate, cellulose acetate hexahydrophthalate, hydroxypropyl methylcellulose hexahydrophthalate, hydroxypropyl methylcellulose phthalate (HPMCP), cellulose propionate phthalate, cellulose acetate maleate, cellulose acetate trimellitate (CAT), cellulose acetate butyrate, cellulose acetate propionate, methacrylic acid/methacrylate polymer (acid number 300 to 330 commercially sold under the tradename EUDRAGIT L™, which is an anionic copolymer based on methacrylate and available as a powder (also known as methacrylic acid copolymer, type A (USP NF), methacrylic acid-methyl methacrylate copolymer, ethyl methacrylate-methylmethacrylate-chlorotrimethylammonium ethyl methacrylate copolymer, poly(methacrylate ethylacrylate) (1:1) copolymer (MA-EA), poly(methacrylate methylmethacrylate) (1:1) copolymer (MA-MMA), poly(methacrylate methylmethacrylate) (1:2) copolymer, Eudragit L-30-D™ (MA-EA, 1:1), Eudragit™L-100-55™ (MA-EA, 1:1), hydroxypropylmethylcellulose acetate succinate (HPMCAS), AQUACOAT™ (HBPMCAS) and the like and combinations and mixtures thereof. Other examples include natural resins, such as shellac, Sandarac™, copal collophorium and mixtures thereof. Examples of synthetic resin bearing carboxyl groups include methacrylic acid: acrylic acid ethyl ester 1:1 copolymer solid substance of the acrylic dispersion commercially available under the tradename Eudragit L-100-55. Other enteric coating polymers known in the art, include for example hydroxypropyl methylcellulose phthalate HP50 (HPMCP-HP50) (USP/NF 220824), HP55 (HPMCP-HP55) (USP/NF type 200731) and HP55S available from Shin Etsu Chemical, Coateric™ (polyvinyl acetate phthalate available from Colorcon Ltd.), Sureteric™ (polyvinyl acetate phthalate available from Colorcon, Ltd.), or Aquateric™ (cellulose acetate phthalate available from FMC Corp.), and the like, and may be employed. The enteric coating will also preferably contain a plasticizer which is preferably diethyl phthalate, although the invention is not limited in this respect and other plasticizers may be used such as triethyl citrate (Citroflex-2), triacetin, tributyl sebecate, or polyethylene glycol. Other coating examples include beeswax and glyceryl monostearate; beeswax, shellac and cellulose; and cetyl alcohol, mastic and shellac (disclosed in U.S. Pat. No. 5,225,202), shellac and stearic acid (disclosed in U.S. Pat. No. 2,809,918); polyvinyl acetate and ethyl cellulose (disclosed in U.S. Pat. No. 3,835,221); and a neutral copolymer of polymethacrylic acid esters containing metallic stearates (disclosed in U.S. Pat. Nos. 4,728,512 and 4,794,001).

The enteric polymer is present in the coating of the methenamine compound in appropriate amounts as determinable by one skilled in the art, such as ranging from about 1% to about 25% by weight of the coating, more preferably from about 2% to about 25% by weight and even more preferably from about 2% to about 20% and especially from about 5% to about 20% and even more especially from about 5% to about 15% by weight of the coat and most preferably from about 8% to about 15% by weight of the coat. The enteric polymer, however, is preferably not present in amounts greater than 25% by weight of the coat. Depending upon the composition and/or thickness, the enteric coatings are resistant to stomach acid for required periods of time before they begin to disintegrate and permit slow release of the drug in the lower stomach or upper part of the small intestines. The coated methenamine compound provides a delayed release of the methenamine compound which is soluble or erodible in intestinal juices, substantially pH neutral or basic fluids but for the most part insoluble in gastric juices or acidic fluids.

In combination with the coated methenamine compound, an acidifier that is effective to acidify the patient's urine to below a pH of 6 is used. More preferably the acidifier causes a urinary pH of from about 3 to about 6, and still more preferably of from about 4 to about 5. Representative acidifiers includes for example, without limitation, sodium biphosphate, potassium biphosphate, ammonium chloride, methionine, ammonium bisphosphate, amino acid, citric acid, fumaric acid and other alpha hydroxy acids, such as ascorbic acid and other like solid acids. Unit dosage amounts of the acidifier includes those amounts effective for reducing the urinary pH, with representative amounts ranging from about 1 gram or less, such as about 500 mg or less including from about 200 mg to about 500 mg, more preferably from about 300 mg to about 500 mg, still more preferably from about 400 mg to about 500 mg, and most preferably about 500 mg. Representative preferred unit dosages include about 500 mg of methenamine compound. In a preferred embodiment, the methenamine compound is present in with about equal amounts of the acidifier, such as an amount of about 350 mg of methenamine in combination with about 350 mg of acidifier, or other pharmaceutically effective equal amounts such as about 300 mg, 325 mg, 375 mg, 400 mg, 425 mg, 450 mg, 475 mg, 500 mg, 525 mg, etc., and amounts therebetween, of both the methenamine and acidifier. In another preferred embodiment, different amounts of the methenamine and acidifier may be used, such as for example, without limitation, combinations of the above-listed amounts, e.g., 400 mg of methenamine compound and 450 mg of acidifier, 500 mg of methenamine compound and 425 mg of acidifier, and the like.

The methenamine compound API may be used as a single active agent, or may be combined with other active agents, vitamins, minerals, dietary supplements, etc. The medicament may include a second, or more, active pharmaceutical ingredients. The second active pharmaceutical ingredient may include antiseptic agent such as azo dyes, pyridium, methylene blue, antispasmodis, phenyl salicylate, benzoic acid, etc., parasympatholytics such as atropine sulfate, hyoscyamine sulfate, and the like, and combinations thereof. Other pharmaceutical actives suitable for inclusion with the present invention include, for example without limitation, analgesics, such as aspirin, acetaminophen, etc. Preferably, the second pharmaceutical ingredient includes at least one antiseptic agent. Preferred therapeutic combinations of these pharmaceutical actives include, but are not limited to combination of, methenamine and methylene blue, methenamine and benzoic acid, methenamine mandelate and methylene blue, methenamine mandelate and benzoic acid, methenamine hippurate and methylene blue, and methenamine hippurate and benzoic acid.

The medicament may include any appropriate solid dosage form, such as pills, tablets, micronized granulates, controlled release particles and the like. Preferably the medicament is in the form of a tablet, and more preferably the medicament is present in a bi-layer form having an enteric coated methenamine compound pressed on one side of an acidifier with the combination of the methenamine compound and acidifier then encased in a protective coat. Other forms of the final tablet include a coated methenamine compound, effective to inhibit conversion of the methenamine compound to formaldehyde, combined with an amount of acidifier that is further encased in a protective coating for general handling of pharmaceuticals (referred to herein as a “handling coat”), such as forms of completely covering the coated methenamine with the acidifier and applying a handling coat thereon, micronized beads of coated methenamine compound intermixed with acidifier that is placed within a handling coat, etc. The coated methenamine compound together with the acidifier may also be placed within a capsule or other similar unitary holding device for pharmaceutical administration. Preferably, the medicament of the present invention is produced through a tableting process that includes the steps of preparing a coated methenamine compound, and tableting the coated methenamine compound in combination with the acidifier. Preparation of the tablet is accomplished using ingredients of a purity such that it is suitable for pharmaceutical administration to patients. Generally, the pharmaceutical formulation contains at least one conventional pharmaceutical excipient in addition to the acidifier and methenamine compound.

The tablets of the present invention can also comprise additional components such as adsorbent, antioxidant, colorant, flavorant, sweetening agent, tablet antiadherent, tablet binder, tablet and capsule diluent, tablet direct compression excipient, tablet disintegrant, tablet glidant, tablet lubricant, tablet or capsule opaquant and/or tablet polishing agents, and other such tabletting ingredients known in the art that do not interfere with the pharmaceutical effectiveness of the present invention. Adsorbents include agents capable of holding other molecules onto its surface by physical or chemical (chemisorption) means such as powdered and activated charcoal and other such materials known to those of ordinary skill in the art. Antioxidants include agents that inhibit oxidation generally intended to prevent the deterioration of the tablet by the oxidative process such as ascorbic acid, ascorbic palmitate, Vitamin E, butylated hydroxyanisole, butylated hydroxytoluene, hypophosphorous acid, monothioglycerol, propyl gallate, sodium ascorbate, sodium bisulfite, sodium formaldehyde sulfoxylate, sodium metalbisulfite and other such materials known to those of ordinary skill in the art. Antiadherents include agents that prevent the sticking of tablet formulation ingredients to the punches and dies in a tableting machine during production such as magnesium stearate, calcium stearate, talc, glyceryl behenate, poly(ethylene glycol), hydrogenated vegetable oil, mineral oil, stearic acid, combinations thereof and other such materials known to those of ordinary skill in the art. Binders include substances used to cause adhesion of powder particles in tablet granulations such as acacia, alginic acid, tragacanth, carboxymethylcellulose sodium, poly(vinylpyrrolidone), compressible sugar (e.g., NuTab), ethylcellulose, gelatin, liquid glucose, methylcellulose, povidone and pregelatinized starch, combinations thereof and other materials known to those of ordinary skill in the art. When needed, other binders may also be included in the medicament. Exemplary binders include starch, poly(ethylene glycol), guar gum, polysaccharide, bentonites, sugars, invert sugars, poloxamers (PLURONIC™ F68, PLURONIC™ F127), collagen, albumin, celluloses in nonaqueous solvents, combinations thereof and the like. Other binders include, for example, poly(propylene glycol), polyoxyethylene-polypropylene copolymer, polyethylene ester, polyethylene sorbitan ester, poly(ethylene oxide), microcrystalline cellulose, poly(vinylpyrrolidone), combinations thereof and and other such materials known to those of ordinary skill in the art. Diluents or fillers include inert substances used as fillers to create the desired bulk, flow properties, and compression characteristics in the preparation of tablets and capsules such as dibasic calcium phosphate, kaolin, sucrose, mannitol, microcrystalline cellulose, powdered cellulose, precipitated calcium carbonate, sorbitol, starch, combinations thereof and other such materials known to those of ordinary skill in the art. Tablet direct compression excipients include compounds used in direct compression tablet formulations such as dibasic calcium phosphate (e.g., Ditab™), microcrystalline cellulose, direct compression lactose (e.g., Tablettose™, Lactose DT), combinations thereof and other such materials known to those of ordinary skill in the art. Glidants include agents used in tablet and capsule formulations to improve flow-properties during tablet compression and to produce an anti caking effect such as colloidal silica, calcium silicate, magnesium silicate, silicon hydrogel, cornstarch, talc, combinations thereof and other such materials known to those of ordinary skill in the art. Lubricants include substances used in tablet formulations to reduce friction during tablet compression such as calcium stearate, magnesium stearate, mineral oil, stearic acid, zinc stearate, combinations thereof and other such materials known to those of ordinary skill in the art. Tablet opaquants include compounds used to used in tablet coatings or capsules providing useful opacity which can aid the stability to the light in case of sensitive agents (either used alone or in combination with a colorant), such as titanium dioxide and other such materials known to those of ordinary skill in the art. Tablet polishing agents include compounds used to impart brightness to the surface of the coated tablets such as carnauba wax, white wax, combinations thereof and other such materials known to those of ordinary skill in the art. Disintegrants or disintegrators include compounds used in solid dosage forms to promote the disruption of the solid mass into smaller particles which are more readily dispersed or dissolved such as starches such as corn starch, potato starch, pre-gelatinized and modified starches thereof, sweeteners, clays, such as bentonite, microcrystalline cellulose (e.g., Avicel™), carboxymethylcellulose calcium, cellulose polyacrylin potassium (e.g., Amberlite™), alginates, sodium starch glycolate, gums such as agar, guar, locust bean, karaya, pectin, tragacanth, gelatine, combinations thereof and other such materials known to those of ordinary skill in the art. Suitable coloring agents or colorants may be used with such compounds including, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and iron oxide (black, red, yellow), other F.D. & C. dyes and natural coloring agents such as grape skin extract, beet red powder, beta-carotene, annato, carmine, turmeric, paprika, combinations thereof and other such materials known to those skilled in the art. Flavoring agents (herein referred to also as “flavorants”), sweetening agents, and combinations thereof to mask the inherently bitter taste associated with the acidic medicament of the present invention, and thereby improving patient compliance for taking such medicament. Flavorants are used to impart a pleasant flavor and often odor to a pharmaceutical preparation. Suitable flavoring agents include natural and artificial flavors, such as synthetic flavor oils and flavoring aromatics and/or natural oils, extracts from plants, leaves, flowers, fruits and so forth and combinations thereof. Representative suitable flavoring agents may be for example, without limitation, menthol, cinnamon, wintergreen, clove, bay, anise, eucalyptus, thyme, cedar leave, nutmeg, sage, bitter almonds and cassia, vanilla, artificial vanilla, chocolate, artificial chocolate, bubble gum, both natural and artificial fruit flavors, such as cherry flavor, grape flavor, orange flavor, strawberry flavor, lemon flavor, grapefruit flavor and Amint@ flavors such as peppermint flavor and spearmint flavor, lime flavor, apple flavor, pear flavor, peach flavor, raspberry flavor, plum flavor, pineapple flavor, apricot flavor and so forth, including combinations of two or more thereof. Flavoring agents are generally provided as a minor component of the solid pharmaceutical formulation in amounts effective to provide a palatable flavor to the solid pharmaceutical formulation. The amount of flavoring agent may depend on a number of factors, including the desired organoleptic effect. The precise amount of sweetening and/or flavoring agent(s) depends on the properties of the agent(s) used, however generally in an amount that is sufficient to mask the bitter taste associated with the acidic medicament as determinable by one skilled in the art. However, flavoring agents are generally present in the solid pharmaceutical formulation in amounts in the range of from about 0 grams to about 10 mg per tablet, with preferred amounts of from about 2 mg to about 5 mg. Sweeteners suitable for inclusion in the present invention may be determined by one skilled in the art including, for example without limitation, both natural and artificial sweeteners such as the representative sweetening agents of intense sweeteners such as sorbitol, sucrose, saccharins such as sodium saccharin, cyclamates such as sodium cyclamates, aspartame, sucralose, thaumatin, acesulfam K, and the like, and sugars such as monosaccharides, disaccharides and polysaccharides. Representative sugars useful in the present invention include, without limitation, xylose, ribose, glucose, mannose, galactose, fructose, dextrose, sucrose, maltose, partially hydrolyzed starch or corn syrup, and sugar alcohols such as sorbitol, xylitol, mannitol, glycerin, etc. and combination thereof. Sugar sweeteners may be replaced or augmented by water soluble artificial sweeteners, such as the suitable artificial sweeteners previously listed and mixtures thereof. The amount of artificial sweetener used in the solid pharmaceutical formulation may vary to provide an appropriate amount of sweetness to the medicament as determinable by one skilled in the art, generally in amounts similar to those of sugar sweeteners described above. Mixtures of sweetening and/or flavoring agents are preferably used. Other excipients may be used as needed, and several the excipients previously identified are understood to be used in the art of pharmaceutical formulations to serve a variety of functions or purposes.

The above-described medicament is used as a urinary antiseptic treatment. With the dosing of a patient with the medicament, the medicament provides an antiseptic affect. The medicament is useful in the suppression or elimination of bacteriuria associated with chronic and recurrent infections of the urinary tract, including pyelitis, pyelonephritis, cystitis and infected residual urine accompanying neurogenic bladder or other causes. This includes relief of discomfort of the lower urinary tract caused by hypermotility resulting from inflammation or diagnostic procedures and in the treatment of cystitis, urethritis and trigonitis when caused by organisms which are susceptible to formaldehyde.

The present invention provides methods of treating a subject (e.g., mammal, particularly humans) comprising administering to a subject in need of such treatment a therapeutically effective amount of at least one active ingredient, formulation thereof, or unit dose forms thereof, each as described herein.

As used herein, the term “treatment”, or a derivative thereof, contemplates partial or complete inhibition of the stated disease state such as, for example, bacteriuria associated with chronic and recurrent infections of the urinary tract, when an active ingredient of the present invention is administered prophylactically or following the onset of the disease state for which such active ingredient of the present invention is administered. For the purposes of the present invention, “prophylaxis” refers to administration of the active ingredient(s) to a mammal to protect the mammal from any of the disorders set forth herein, as well as others.

The typical active daily dose of the methenamine compound of the present invention depends on various factors such as, for example, the individual requirement of each patient in light of age, weight, etc., the type of infection or disease, and other like considerations determinable by those skilled in the medical arts. An attending physician may adjust the dosage rate based on these and other criteria if he or she so desires. As an example, a suitable oral dosage form may encompass from about 500 mg of methenamine and 500 mg of acidifier total daily dose, typically administered in one single dose or equally divided doses. It should be appreciated that other distinct daily doses may be administered to a subject, as appreciated by an attending physician.

The phrase “pharmaceutically acceptable” is employed herein to refer to those compounds, materials, compositions, and/or dosage forms which are, within the scope of sound medical judgment, suitable for use in contact with tissues of human beings and animals and without excessive toxicity, irritation, allergic response, or any other problem or complication, commensurate with a reasonable benefit/risk ratio.

The amount of therapeutic compound incorporated in each device of the invention will be at least one or more dosage form and can be selected according to known principles of pharmacy. An effective amount of therapeutic compound is specifically contemplated. By the term “effective amount”, it is understood that, with respect to, for example, pharmaceuticals, a pharmaceutically effective amount is contemplated. A pharmaceutically effective amount is the amount or quantity of a drug or pharmaceutically active substance which is enough for the required or desired therapeutic response, or in other words, the amount, which is sufficient to elicit an appreciable biological response when, administered to a patient. The appreciable biological response may occur as a result of administration of single or multiple unit doses of an active substance. Depending upon the active substance used and upon the amount of active substance present in a particular device according to the invention, a unit dose may comprise one or more such devices.

Formulations of the solid pharmaceutical medicament of the present invention are illustrated in the examples below.

### EXAMPLE 1 (PROPHETIC)

500 mg amounts of solid methenamine are coated with a delayed-release coating. The coated methenamine units are tableted with 500 mg of sodium biphosphate in an oral dosage form.

### EXAMPLE 2 (PROPHETIC)

1 gram amounts of methenamine hippurate are coated with glaze covering and then tableted with 1 gram units of sodium biphosphate.

### EXAMPLE 3 (PROPHETIC)

750 mg amounts of methenamine mandelate are combined with methylene blue and coated with a delay-release coating. The coated methenamine mandelate/methylene blue tableted with 1 gram units of methionine.

### EXAMPLE 4 (PROPHETIC)

350 mg of methenamine mandelate is enteric coated and then combined with 350 mg of sodium biphosphate in a bi-layer tablet.

The foregoing summary, description, and examples of the invention are not intended to be limiting, but are only exemplary of the inventive features which are defined in the claims.

What is claimed is:

1. A solid oral pharmaceutical medicament, comprising:
a coated methenamine compound effective to delay release of the methenamine within the gastrointestinal tract; in combination with,
an urinary acidifier.

2. The medicament of claim 1, wherein the methenamine compound is selected from the group consisting of methenamine, methenamine salts and combinations thereof.

3. The medicament of claim 2, wherein the methenamine compound is methenamine.

4. The medicament of claim 2, wherein the methenamine salts are selected from the group consisting of methenamine mandelate and methenamine hippurate.

5. The medicament of claim 4, wherein the methenamine salt is methenamine mandelate.

6. The medicament of claim 4, wherein the methenamine salt is methenamine hippurate.

7. The medicament of claim 1, wherein the coated methenamine compound comprises a delay-release coating.

8. The medicament of claim 1, wherein the urinary acidifier is effective to acidify urine to a pH of from about 3 to about 6.

9. The medicament of claim 1, wherein the acidifier is selected from the group consisting of sodium biphosphate, potassium biphosphate, ammonium chloride, methionine, ammonium bisphosphate, amino acid, citric acid, fumaric acid and ascorbic acid.

10. The medicament of claim 1, wherein the medicament form is selected from the group consisting of pill, tablet, micronized granulates and controlled release particles.

11. The medicament of claim 12, wherein the medicament form is a pill.

12. The medicament of claim 12, wherein the medicament form is a tablet.

13. The medicament of claim 12, wherein the medicament form is micronized granulates.

14. The medicament of claim 12, wherein the medicament form is controlled release particles.

15. A unit dosage comprising the medicament of claim 1.

16. A unit dosage of the medicament of claim 15, wherein the methenamine compound is present in an amount of from about 300 mg to about 2 grams and the acidifier is present in an amount of from about 300 mg to about 1 gram.

17. The medicament of claim 1, further comprising second active pharmaceutical ingredient.

18. The medicament of claim 17, wherein the second active pharmaceutical ingredient comprises an antiseptic agent.

19. The medicament of claim 18, wherein the antiseptic agent is selected from the group consisting of azo dyes, pyridium, methylene blue, antispasmodic, phenyl salicylate, benzoic acid, and combinations thereof.

20. A method for urinary antiseptic treatment, comprising the steps of:
providing the medicament of claim 1; and,
dosing a patient with the medicament.
